(19) United States
(12) Patent Application Publication
(10) Pub. No.: US 2015/0265444 A1
(43) Pub. Date: Sep. 24, 2015

(54) STENT DELIVERY DEVICE

(71) Applicant: TERUMO KABUSHIKI KAISHA, Tokyo (JP)

(72) Inventor: Takashi KITAOKA, Hadano-shi (JP)

(73) Assignee: TERUMO KABUSHIKI KAISHA, Tokyo (JP)

(21) Appl. No.: 14/731,559

(22) Filed: Jun. 5, 2015

Related U.S. Application Data
(63) Continuation of application No. PCT/JP2013/050127, filed on Jan. 8, 2013.

(51) Int. Cl. A61F 2/962; A61F 2/966

(52) U.S. Cl. CPC A61F 2/962; A61F 2/966; A61F 2230/0069

(57) ABSTRACT

A stent delivery device is disclosed, which includes a stent arranged in a self-expandable state between a shaft and an inner sheath. In a state where an outer sheath is moved rearward and an expansion limiting portion is exposed outward, the expansion limiting portion formed in a distal portion of the inner sheath limits expansion of the stent so that an outer diameter of a distal end of the stent becomes a first diameter (D1) which is larger than an inner diameter (Da) of the outer sheath, and in a state where the inner sheath is moved rearward and the stent is exposed outward, the stent expands so that the outer diameter of the distal end of the stent becomes a second diameter (D2) which is larger than the first diameter (D1).

### CROSS-REFERENCES TO RELATED APPLICATIONS

This application is a continuation of International Application No. PCT/JP2013/050127 filed on Jan. 8, 2013, the entire content of which is incorporated herein by reference.

### TECHNICAL FIELD

The present disclosure relates to a stent delivery device for causing a stent to indwell in a living body lumen.

### BACKGROUND DISCUSSION

Conventionally, in order to improve a lesion (stenosis or occluded portion) appearing inside a living body lumen such as blood vessels, biliary ducts, bronchial tubes, esophagi, urethrae, and the like, a stent delivery device for causing a stent to indwell in the lesion has been widely used.

For example, JP-T-2008-529719 discloses a stent delivery device, which includes an inner sheath arranged relative to be movable to the shaft in an axial direction, an outer sheath disposed to be slidable relative to the inner sheath in the axial direction, and a stent received in a self-expandable state between the shaft and the inner sheath, and in which multiple strip-shaped tail portions separated by multiple slits are formed on a distal side of the inner sheath for receiving the stent.

The stent delivery device causes the outer sheath to move rearward relative to the inner sheath, thereby expanding the stent so that the multiple tail portions are pressed against an inner wall surface of the living body lumen. Then, the inner sheath is caused to move rearward relative to the shaft, thereby pulling out the multiple tail portions from a portion between the stent and the inner wall surface of the living body lumen. In this manner, damage to the coating of the stent can be minimized when the stent is deployed into the living body lumen and is caused to indwell in the living body lumen.

Incidentally, when a self-expandable stent is caused to indwell at an indwelling-targeted portion in a living body lumen, if a sheath is moved rearward, the stent can simultaneously expand all at once and indwell at an inner wall surface of the living body lumen. Consequently, the stent is likely to be misaligned with the indwelling-targeted portion as a separated distance is farther between the stent in a received state and the indwelling-targeted portion.

The stent delivery device according to the above-described prior art has the tail portion of the inner sheath which is arranged between the stent and the outer sheath. However, the stent delivery device aims to minimize damage to the coating of the stent. Thus, if the outer sheath is moved rearward, the stent simultaneously expands all at once to reach a position of substantially coming into contact with an inner wall surface of the living body lumen. Consequently, the stent cannot be prevented from being misaligned with the indwelling-targeted portion. In addition, after the stent expands, when the tail portion located between the inner wall surface of the living body lumen and the stent is pulled out, the stent may be misaligned with the indwelling-targeted portion of the living body lumen.

### SUMMARY

In accordance with an exemplary embodiment, a stent delivery device is disclosed which can help prevent a stent from being misaligned with an indwelling-targeted portion, when the self-expandable stent is caused to expand and indwell at the indwelling-targeted portion of a living body lumen.

In accordance with an exemplary embodiment, a stent delivery device is disclosed, which can include a shaft, a tubular inner sheath that is arranged on an outer surface side of the shaft so as to be movable relative to the shaft along an axial direction, a tubular outer sheath that is arranged on an outer surface side of the inner sheath so as to be slidable relative to the inner sheath along the axial direction, and a stent that is received in a self-expandable state between the shaft and the inner sheath. A distal portion of the inner sheath has an expansion limiting portion, which is configured to be more flexible than the outer sheath and by which at least a distal end of the stent is received. In a state where the outer sheath is moved rearward and the expansion limiting portion is exposed outward from the outer sheath, the expansion limiting portion limits expansion of the stent so that an outer diameter of the distal end of the stent becomes a first diameter which is larger than an inner diameter of the outer sheath. In a state where the inner sheath is moved rearward and the stent is exposed outward from the inner sheath, the stent expands so that the outer diameter of the distal end of the stent becomes a second diameter, which is larger than the first diameter.

In accordance with an exemplary embodiment, if the outer sheath is moved rearward and the expansion limiting portion of the inner sheath is exposed outward from the outer sheath, the stent can expand so that the outer diameter of the distal end of the stent becomes the first diameter which is larger than the inner diameter of the outer sheath (first expansion). Then, if the inner sheath is moved rearward and the distal end of the stent is exposed outward from the inner sheath, the stent can expand so that the outer diameter of the distal end of the stent becomes the second diameter, which is larger than the first diameter (second expansion). In accordance with an exemplary embodiment, for example, the stent can expand at two steps in the living body lumen. In this manner, after the stent is delivered to an indwelling-targeted portion of the living body lumen, a position of the stent can be finely adjusted by maintaining a state where the stent is subjected to the first expansion so as to shorten a separated distance between the distal end of the stent and the indwelling-targeted portion. Subsequently, the stent can be subjected to the second expansion. In this manner, the stent can be reliably indwelled at the indwelling-targeted portion. Therefore, when the self-expandable stent expands and indwells at the indwelling-targeted portion of the living body lumen, the stent can be prevented from being misaligned with the indwelling-targeted portion.

In accordance with an exemplary embodiment, the inner sheath may have an inner sheath main body which is disposed on a proximal side relative to the expansion limiting portion and which has greater rigidity than that of the expansion limiting portion. In accordance with an exemplary embodiment, when a total length of the expansion limiting portion is set to L, a total length of the stent in a received state is set to L1, and an outer diameter of the stent in a natural state is set to D, an equation of L≦L1−D/2 (L is less than or equal to L1 minus D/2) may be satisfied.

Here, the stent in the natural state means a stent left as it is without having any restraints at all. According to this configuration, the equation of L≦L1−D/2 is satisfied. Therefore, when the distal end of the stent is subjected to the second expansion, the proximal end of the stent can be located inside the inner sheath main body which has the greater rigidity than that of the expansion limiting portion. In this manner, the distal end of the stent can be subjected to the second expansion so as to have the second diameter in a state where the proximal end of the stent is pressed against and held by the inner surface of the inner sheath main body by using a relatively strong restraining force. Accordingly, the distal end of the stent can reliably come into contact with the indwelling-targeted portion during the second expansion. Therefore, the distal end of the stent can preferably be prevented from being misaligned with the indwelling-targeted portion in the axial direction. In addition, after the distal end of the stent is indwelled at the indwelling-targeted portion, the movement of the stent with respect to the indwelling-targeted portion can be limited. Therefore, the possibility that the stent may be misaligned with the indwelling-targeted portion during the second expansion of the proximal side of the stent can be reduced.

In the above-described stent delivery device, flexibility of the expansion limiting portion may increase continuously or stepwisely toward a distal end.

In accordance with an exemplary embodiment, the flexibility of the expansion limiting portion can increase continuously or stepwisely toward the distal end. Therefore, in a state where the stent is subjected to the first expansion, the distal end within the stent can be moved closest to the indwelling-targeted portion. In this manner, the stent can be efficiently prevented from being misaligned with the indwelling-targeted portion in the axial direction during the second expansion of the distal end of the stent.

The above-described stent delivery device may further include a stopper portion which is disposed on the shaft so as to limit displacement along the axial direction of the stent.

According to this configuration, the stopper portion can be disposed on the shaft portion. Therefore, the stent can be preferably prevented from being misaligned with the indwelling-targeted portion during the expansion of the stent.

In the above-described stent delivery device, the stopper portion may be located inside the inner sheath main body and may engage with the stent in a state where the distal end of the stent is exposed from the expansion limiting portion.

According to this configuration, in a state where the distal end of the stent is exposed from the expansion limiting portion, the stopper portion is located inside the inner sheath main body and engages with the stent. Therefore, the stent can be reliably prevented from being misaligned with the indwelling-targeted portion in the axial direction during the second expansion of the distal end of the stent.

In the above-described stent delivery device, the stopper portion may be in contact with a proximal end of the stent in a state of being fixed to the shaft.

According to this configuration, the stopper portion is in contact with the proximal end of the stent in a state of being fixed to the shaft. Therefore, when the inner sheath is moved rearward relative to the shaft, the stent can be reliably prevented from being misaligned to the proximal side with the indwelling-targeted portion.

In the above-described stent delivery device, the stopper portion may have a locking portion which extends in a radial direction of the shaft and which is arranged to be inserted into a gap of struts of the stent.

According to this configuration, the locking portion of the stopper portion can be inserted into the gap of the struts of the stent. Therefore, when the inner sheath is moved rearward relative to the shaft, the stent can be reliably prevented from being misaligned with the indwelling-targeted portion in the axial direction. The stent expands so that each of the struts slips out from the locking portion of the stopper portion. Therefore, the possibility that the stopper portion may interfere with the expansion of the stent in the radial direction may be reduced.

### BRIEF DESCRIPTION OF THE DRAWINGS

FIG. 1 is a partially omitted vertical cross-sectional view of a stent delivery device according to an exemplary embodiment of the present disclosure.

FIG. 2 is an enlarged vertical cross-sectional view on a distal side of the stent delivery device in FIG. 1.

FIG. 3 is a cross-sectional view taken along line III-III in FIG. 2.

FIG. 4 is a side view of a stent in a natural state.

FIG. 5 is a partially omitted vertical cross-sectional view for illustrating a state where the stent in FIG. 1 is subjected to a first expansion.

FIG. 6 is a partially omitted vertical cross-sectional view for illustrating a state where a distal end of the stent in FIG. 5 is subjected to a second expansion.

FIG. 7 is a partially omitted vertical cross-sectional view for illustrating a state where the stent in FIG. 6 completely indwells at an indwelling-targeted portion of a living body lumen.

FIG. 8A is a partially omitted vertical cross-sectional view for illustrating a first modification example of an expansion limiting portion configuring the stent delivery device.

FIG. 8B is a partially omitted vertical cross-sectional view for illustrating a second modification example of the expansion limiting portion configuring the stent delivery device.

FIG. 9A is a partially omitted vertical cross-sectional view for illustrating a modification example of a stopper portion configuring the stent delivery device.

FIG. 9B is a cross-sectional view taken along line IXB-IXB in FIG. 9A.

### DETAILED DESCRIPTION

Hereinafter, a stent delivery device according to the present embodiment will be described in detail with reference to preferred embodiments and the accompanying drawings.

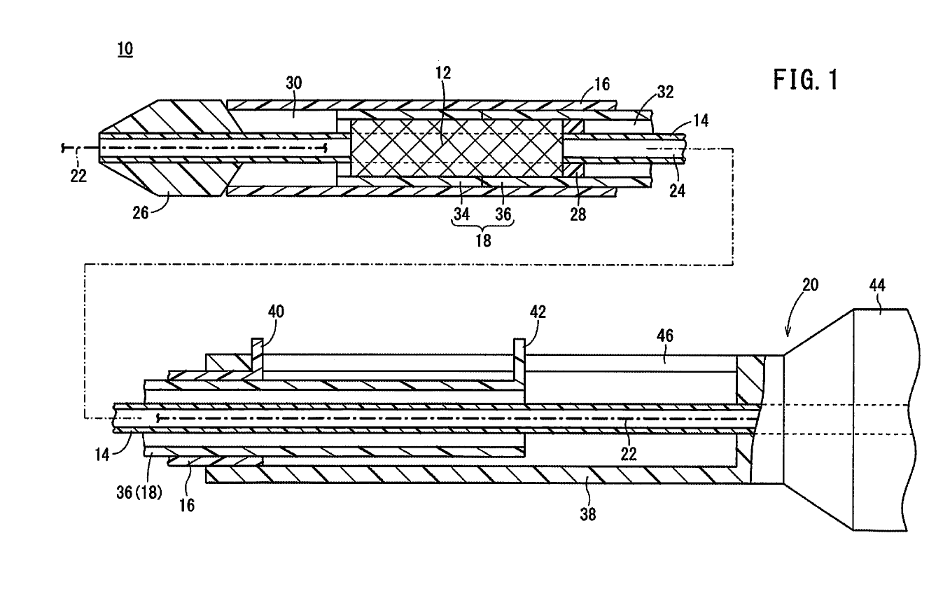

A stent delivery device 10 (hereinafter, simply referred to as a “delivery device 10”) according to an embodiment of the present disclosure is a medical device for causing a stent 12 to indwell in a lesion in order to improve the lesion appearing inside a living body lumen such as blood vessels, biliary ducts, bronchial tubes, esophagi, urethrae, and the like. In the following description, the left side (stent 12 side) of the delivery device 10 in FIG. 1 is referred to as a “distal” side, and the right side (hub 44 side) of the delivery device 10 is referred to as a “proximal (rear)” side.

As illustrated in FIG. 1, the delivery device 10 can include a stent 12, an elongated shaft 14 having a thin diameter, an inner sheath 18 arranged on an outer surface side of the shaft 14 so as to be movable relative to the shaft 14 along an axial direction, an outer sheath 16 arranged on an outer surface side of the inner sheath 18 so as to be slidable relative to the inner sheath 18 along the axial direction, and a handle portion 20 configuring a proximal portion of the delivery device 10.

The stent 12 delivered into and caused to indwell in the living body lumen has a self-expandable function, and is received by a space formed between the shaft 14 and the inner sheath 18, thereby being brought into a state where the stent 12 is folded by the expansion being limited (contraction state, self-expandable state). The stent 12 can employ a configuration of axially arraying multiple frames in which a wire made of super-elastic alloy or the like such as Ti—Ni alloy and the like is formed in a ring shape or a Z-shape, or a configuration of braiding the wires made of the super-elastic alloy or the like in a mesh shape.

The shaft 14 is a flexible tubular member in which a guidewire lumen 24 into which a guidewire 22 is inserted is formed to have a through-hole over the total length of the shaft 14. The guidewire 22 is used to guide the delivery device 10 including the shaft 14 and the like to a lesion of the living body lumen. The shaft 14 extends to the proximal end of the handle portion 20 in a state of being arranged inside the outer sheath 16 so as to protrude beyond the distal end of the outer sheath 16.

A nose portion (nose cone) 26 is disposed in the distal end of the shaft 14. The nose portion 26 has a distal portion whose diameter decreases in a tapered shape toward the distal side, an intermediate portion whose diameter is constant, and a proximal portion whose diameter decreases in a tapered shape toward the proximal side. An outer diameter dimension of the intermediate portion of the nose portion 26 is set to be larger than an inner diameter of the outer sheath 16. In accordance with an exemplary embodiment, the nose portion 26 functions as a stopper for limiting movement toward the distal side of the outer sheath 16.

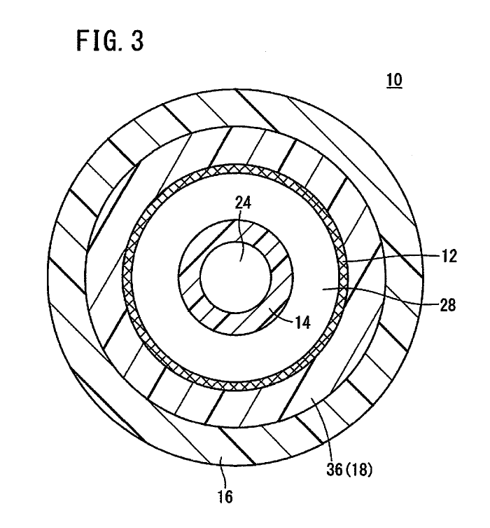

A stopper portion 28 for limiting displacement toward the proximal side of the stent 12 received inside the inner sheath 18 is disposed on the distal side of the shaft 14. The stopper portion 28 can be fixedly attached to an outer surface of the shaft 14 in a state of being in contact with the proximal end of the stent 12. In addition, for example, the stopper portion 28 can be configured to have an annular shape (refer to FIG. 3).

In accordance with an exemplary embodiment, the outer sheath 16 is a flexible tubular member in which the lumen 30 enabling the inner sheath 18 to be arranged therein is formed to have a through-hole over the total length. The outer sheath 16 is arranged so as to be slidable relative to the inner sheath 18 in the axial direction, and can have rigidity to such an extent so as not to be deformed due to an expansion force (spring force) of the stent 12 received inside the inner sheath 18.

A configuration material of the outer sheath 16 is not particularly limited, and may include polyolefin such as polyethylene, polypropylene, and the like, polyamide, polyester such as polyethylene terephthalate, and the like, fluorinated polymers such as PTFE, ETFE, and the like, thermoplastic elastomer such as polyamide elastomer, polyester elastomer, and the like, stainless steel, super-elastic metal, and the like.

The inner sheath 18 can be a flexible tubular member in which a lumen 32 enabling the shaft 14 to be arranged therein is formed to have a through-hole over the total length of the inner sheath 18. The inner sheath 18 can be arranged inside the outer sheath 16 so that the distal end of the inner sheath 18 is located on the proximal side relative to the distal end of the outer sheath 16, and has an expansion limiting portion 34 configuring the distal portion of the inner sheath 18 and an inner sheath main body 36 disposed on the proximal side relative to the expansion limiting portion 34.

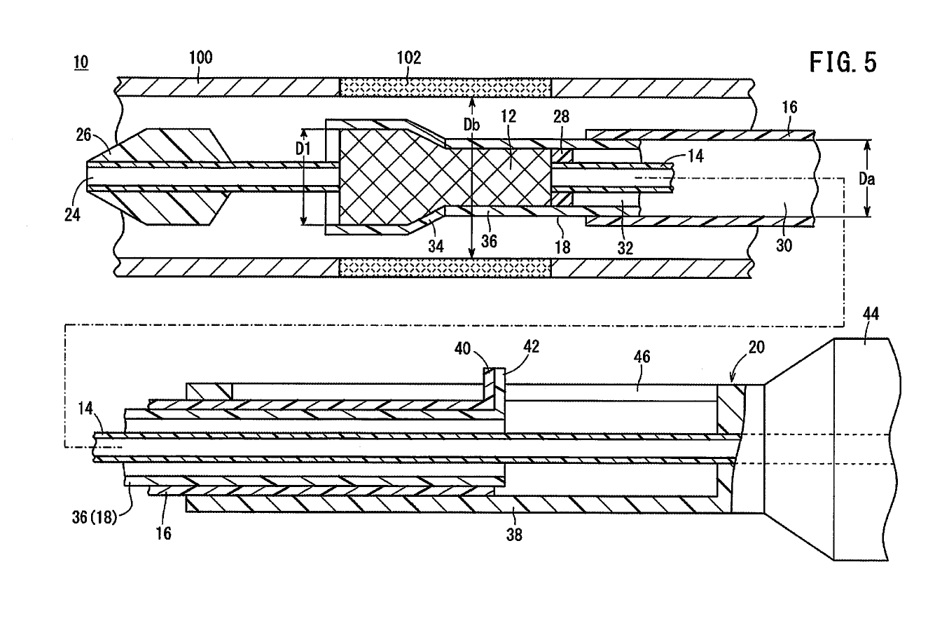

In accordance with an exemplary embodiment, the expansion limiting portion 34 can be used to limit the stent 12 so as not to expand all at once when the stent 12 is discharged (released) to the living body lumen, and at least the distal end of the stent 12 is received into the expansion limiting portion 34. In addition, the expansion limiting portion 34 can be configured to be more flexible than the outer sheath 16. For example, the expansion limiting portion 34 can be configured to be expandable to have a first diameter D1 which is larger than an inner diameter Da of the outer sheath 16 by using an expansion force of the stent 12, in a state where the outer sheath 16 is moved rearward to the proximal side relative to the inner sheath 18 and the expansion limiting portion 34 is exposed outward from the outer sheath 16 (refer to FIG. 5).

In accordance with an exemplary embodiment, for example, the expansion limiting portion 34 can be configured to include a material which is the same as that of the outer sheath 16. In this case, if the expansion limiting portion 34 is formed to be thinner than the outer sheath 16 (if the thickness of the expansion limiting portion 34 is formed to be thinner than the thickness of the outer sheath 16), the expansion limiting portion 34 can be more flexible than the outer sheath 16. For example, the expansion limiting portion 34 may be configured to be more flexible than the outer sheath 16 by changing the configuration material of the expansion limiting portion 34 and the configuration material of the outer sheath 16.

In accordance with an exemplary embodiment, the inner sheath main body 36 can be configured to have rigidity which is greater than that of the expansion limiting portion 34, and can be configured to include a material which is the same as that of the outer sheath 16, for example. In addition, for example, the inner sheath main body 36 can be configured to include a material, which can be the same as that of the expansion limiting portion 34. In this case, for example, if the inner sheath main body 36 is formed to be thicker than the expansion limiting portion 34 (if the thickness of the inner sheath main body 36 is formed to be thicker than the thickness of the expansion limiting portion 34), the inner sheath main body 36 can be configured to have rigidity which is greater than that of the expansion limiting portion 34. In this case, for example, the expansion limiting portion 34 and the inner sheath main body 36 may be configured integrally with each other. For example, the inner sheath main body 36 may be configured to have the rigidity, which is greater than that of the expansion limiting portion 34 by changing the configuration material of the expansion limiting portion 34 and the configuration material of the inner sheath main body 36.

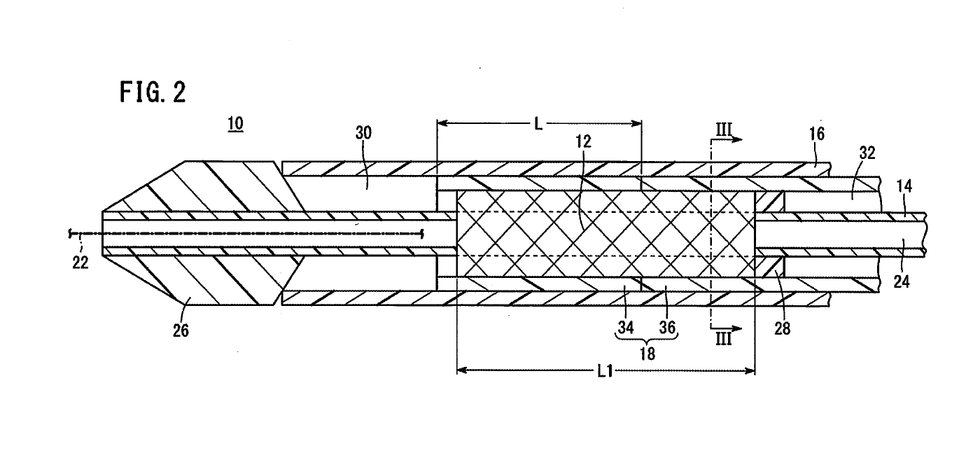

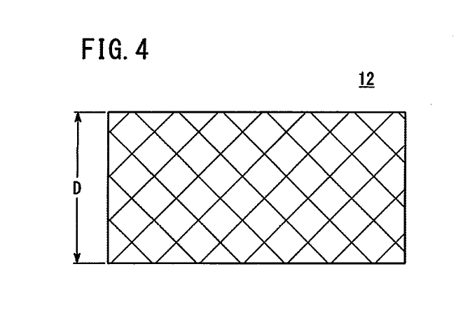

As illustrated in FIGS. 2 and 4, the delivery device 10 according to the present embodiment satisfies an equation of L≦L1−D/2, when a total length of the expansion limiting portion 34 is set to L, a total length of the stent 12 in a received state is set to L1, and an outer diameter of the stent 12 in a natural state is set to D. In accordance with an exemplary embodiment, the stent 12 in the natural state means the stent 12 left as it is without having any restraints at all (non-restrained state). For example, the stent 12 in the natural state is in a state of being left as it is without any restraints in the axial direction and the radial direction.

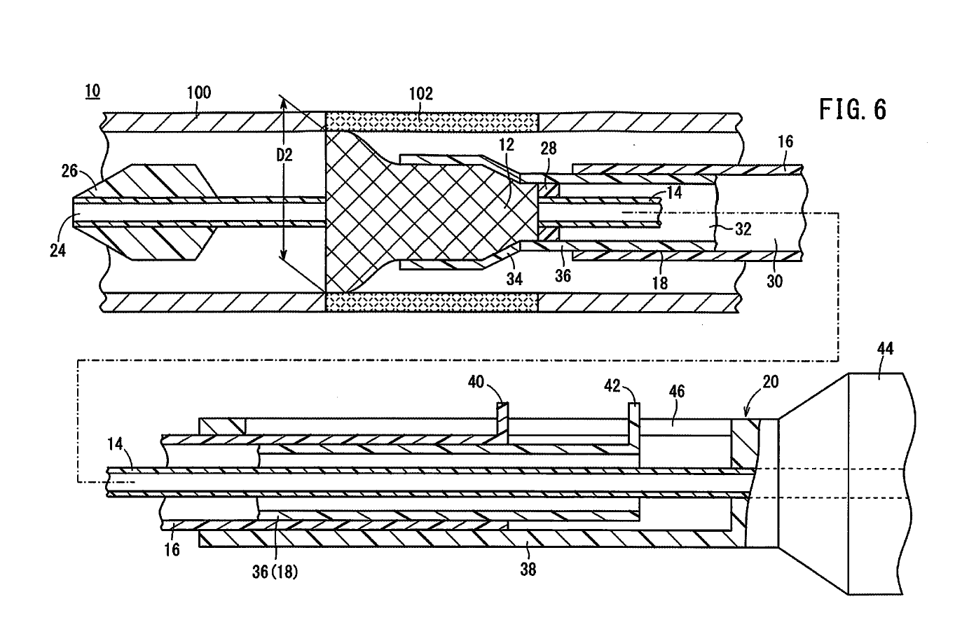

In accordance with an exemplary embodiment, the delivery device 10 satisfying the above-described equation, in the stent 12 in a received state, the proximal end of the stent 12 is located inside the inner sheath main body 36. Then, in a state where the inner sheath 18 is moved rearward to the proximal side relative to the shaft 14 and the distal end of the stent 12 is exposed outward from the expansion limiting portion 34, that is, in a state where the distal end of the stent 12 expands and indwells in the living body lumen, the proximal end of the stent 12 can be brought into contact with the stopper portion 28 inside the inner sheath main body 36 (refer to FIG. 6).

The handle portion 20 can have a support portion 38 formed in a tubular shape, an operation pin 40 disposed at the proximal end of the outer sheath 16, an operation pin 42 disposed at the proximal end of the inner sheath main body 36, and a hub 44 fixed to the proximal end of the support portion 38. An opening 46 extending along the axial direction can be formed on an outer surface of the support portion 38, and the respective operation pins 40 and 42 are exposed outward from the support portion 38 via the opening 46. In this manner, for example, a user or the like can move the outer sheath 16 and the inner sheath 18 independently in the axial direction by pinching the respective operation pins 40 and 42 with the user's fingers and causing the respective operation pins 40 and 42 to slide in the axial direction.

The delivery device 10 according to the present embodiment can be basically configured as described above. Hereinafter, an operation and effect thereof will be described.

First, for example, a form of a lesion appearing in a blood vessel 100 (living body lumen) is identified by using an intravascular angiography method or an intravascular ultrasound diagnosis method. Next, for example, a Seldinger technique can be used to percutaneously guide the guidewire 22 into the blood vessel 100 beforehand through the femoral region or the like. The guidewire 22 is inserted into the proximal end from the distal end of the guidewire lumen 24 of the shaft 14. The delivery device 10 including the shaft 14 is guided into the aorta.

Then, under X-ray contrast using an X-ray opaque marker (not illustrated) disposed in the stent 12, the delivery device 10 is allowed to proceed until the stent 12 received in the distal side of the inner sheath 18 reaches an indwelling-targeted portion 102 of the blood vessel 100. Here, the indwelling-targeted portion 102 means a portion where the stent 12 in the blood vessel 100 is indwelled, and for example, means a portion where a lesion is present.

Subsequently, as illustrated in FIG. 5, a user moves the outer sheath 16 to the proximal side relative to the inner sheath 18 by pinching the operation pin 40 with the user's fingers and pulling the operation pin 40 to the proximal side. Then, the expansion limiting portion 34 exposed outward from the outer sheath 16 is configured to be more reasonably flexible than the outer sheath 16. Accordingly, the stent 12 expands (first expansion) so that the outer diameter of the distal end of the stent 12 becomes the first diameter D1 which is larger than the inner diameter Da of the outer sheath 16.

At this time, the first diameter D1 is smaller than a hole diameter Db of the indwelling-targeted portion 102, and the inner sheath 18 is separated from an inner wall surface of the blood vessel 100. The first expansion of the stent 12 in this way can maintain a state where a separated distance is shortened between the distal end of the stent 12 and the indwelling-targeted portion 102. At this stage, the distal end of the outer sheath 16 is located on the proximal side relative to the expansion limiting portion 34, and the expansion limiting portion 34 is completely exposed outward from the outer sheath 16.

Next, a user moves the delivery device 10 in the axial direction, thereby finely adjusting a position of the stent 12 so that the stent 12 reliably indwells at the indwelling-targeted portion 102. Subsequently, the user pinches the operation pin 42 and pulls the operation pin 42 to the proximal side, thereby moving the inner sheath 18 rearward to the proximal side relative to the shaft 14 by a predetermined amount.

Then, as illustrated in FIG. 6, displacement toward the proximal side of the stent 12 is limited by the stopper portion 28. Accordingly, the distal end of the stent 12 is exposed outward from the inner sheath 18. Then, the distal end of the stent 12 exposed outward from the inner sheath 18 expands (second expansion) so as to have a second diameter D2 (D2 is the same as, or is slightly smaller than an outer diameter D of the stent 12 in a natural state) which is larger than the first diameter D1, and indwells at the indwelling-targeted portion 102. Here, the second diameter D2 is substantially as large as the hole diameter Db of the indwelling-targeted portion 102.

At this time, the proximal end of the stent 12 is in contact with the stopper portion 28 inside the inner sheath main body 36. Accordingly, the stent 12 can be prevented from being misaligned with the indwelling-targeted portion 102 in the axial direction. Therefore, the distal end of the stent 12 can be reliably indwelled at a predetermined position of the indwelling-targeted portion 102.

Subsequently, if the inner sheath 18 is further moved rearward to the proximal side relative to the shaft 14, the proximal end of the stent 12 is subjected to the first expansion and is separated from the stopper portion 28. However, since the distal end of the stent 12 has already indwelled at the indwelling-targeted site 102, the possibility that the stent 12 may be misaligned with the indwelling-targeted portion 102 in the axial direction can be reduced.

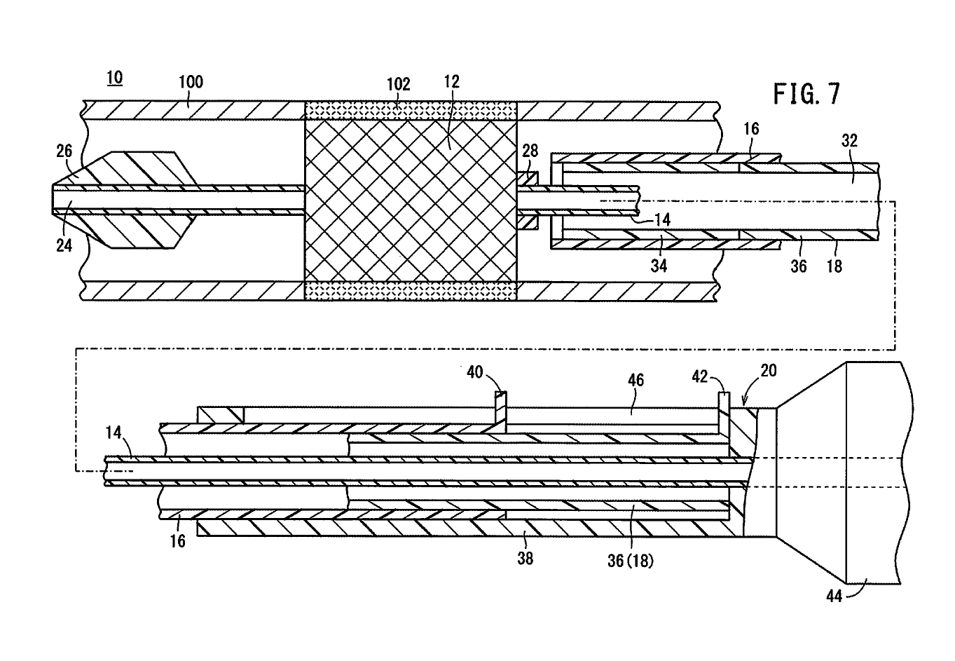

Then, the proximal end of the stent 12 is subjected to the second expansion, thereby causing all portions of the stent 12 to indwell at the indwelling-targeted portion 102 (refer to FIG. 7). At this time, the expansion limiting portion 34 is received inside the outer sheath 16 while contracting inward in the radial direction.

As described above, according to the delivery device 10 of the present embodiment, if the outer sheath 16 is moved rearward and the expansion limiting portion 34 of the inner sheath 18 is exposed outward from the outer sheath 16, the stent 12 is subjected to the first expansion so that the outer diameter of the distal end of the stent 12 becomes the first diameter D1 which is larger than the inner diameter Da of the outer sheath 16. If the inner sheath 18 is moved rearward and the distal end of the stent 12 is exposed outward from the inner sheath 18, the stent 12 is subjected to the second expansion so that the outer diameter of the distal end of the stent 12 becomes the second diameter D2 which is larger than the first diameter D1.

In accordance with an exemplary embodiment, for example, this delivery device 10 can enable the stent 12 to expand at two steps in the blood vessel 100. In this manner, after the stent 12 is delivered to the indwelling-targeted portion 102, a position of the stent 12 can be finely adjusted by maintaining a state where the stent 12 is subjected to the first expansion and the separated distance is shortened between the distal end of the stent 12 and the indwelling-targeted portion 102.

Subsequently, the stent 12 is subjected to the second expansion, thereby enabling the stent 12 to reliably indwell at the indwelling-targeted portion 102. Therefore, when the self-expandable stent 12 expands and indwells at the indwelling-targeted portion 102 in the blood vessel 100, the stent 12 can be prevented from being misaligned with the indwelling-targeted portion 102.

The delivery device 10 according to the present embodiment is configured to satisfy the equation of L≦L1−D/2. Therefore, when the distal end of the stent 12 is subjected to the second expansion, the proximal end of the stent 12 can be positioned inside the inner sheath main body 36 whose rigidity is greater than the expansion limiting portion 34. In addition, during the second expansion, until the inner sheath 18 is moved rearward by a distance corresponding to a radius (D/2) of the stent 12 in at least a natural state, the proximal end of the stent 12 can be held in the inner surface of the inner sheath main body 36.

In this manner, the distal end of the stent 12 can be subjected to the second expansion so as to have the second diameter D2 in a state where the proximal end of the stent 12 is pressed against and held by the inner surface of the inner sheath main body 36 by using a relatively strong restraining force (in a state where there is no restraint of the blood vessel 100, if the inner sheath 18 is moved rearward by the distance corresponding to D/2, the stent 12 expands so as to have the outer diameter D). Accordingly, the distal end of the stent 12 can reliably come into contact with the indwelling-targeted portion 102 during the second expansion. Therefore, the distal end of the stent 12 can be prevented from being misaligned with the indwelling-targeted portion 102.

In addition, after the distal end of the stent 12 indwells at the indwelling-targeted portion 102, the movement of the stent 12 relative to the indwelling-targeted portion 102 is limited by the distal end of the stent 12. Therefore, the possibility that the stent 12 may be misaligned with the indwelling-targeted portion 102 during the second expansion of the proximal side of the stent 12 can be reduced.

The delivery device 10 according to the present embodiment has the stopper portion 28 for limiting displacement along the axial direction of the stent 12 disposed in the shaft 14. Therefore, when the stent 12 expands and indwells at the indwelling-targeted portion 102 of the blood vessel 100, the stent 12 can be prevented from being misaligned with the indwelling-targeted portion 102.

In a state where the distal end of the stent 12 is exposed from the expansion limiting portion 34, the stopper portion 28 is positioned inside the inner sheath main body 36 and is in contact with (engages with) the proximal end of the stent 12. Therefore, the stent 12 can be reliably prevented from being misaligned with the indwelling-targeted portion 102 during the second expansion of the distal end of the stent 12.

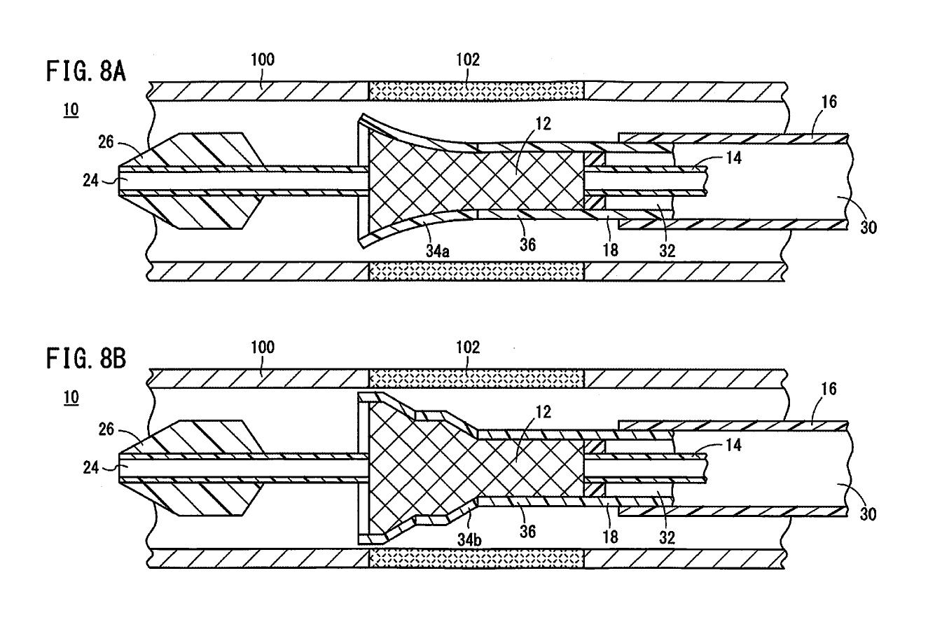

The present embodiment is not limited to the above-described configurations. For example, as illustrated in FIG. 8A, the delivery device 10 according to the present embodiment may have an expansion limiting portion 34a according to a first modification example instead of the expansion limiting portion 34.

The expansion limiting portion 34a is configured so that flexibility thereof continuously increases toward a distal end of the expansion limiting portion 34a. In this case, for example, a component ratio of the configuration material of the expansion limiting portion 34a is changed along the axial direction, and the flexibility can continuously increase toward the distal end of the expansion limiting portion 34a. In addition, the flexibility may continuously increase toward the distal end of the expansion limiting portion 34a by forming the expansion limiting portion 34a so as to be gradually thinner toward the distal end of the expansion limiting portion 34a.

According to the delivery device 10 including the expansion limiting portion 34a of the first modification example, a separated distance between the distal end of the stent 12 and the indwelling-targeted portion 102 can be efficiently shortened in a state where the stent 12 is subjected to the first expansion. In this manner, the distal end of the stent 12 can be reliably indwelled at a predetermined position of the indwelling-targeted portion 102 during the second expansion of the distal end of the stent 12.

In addition, as illustrated in FIG. 8B, the delivery device 10 according to the present embodiment may have an expansion limiting portion 34b according to a second modification example instead of the expansion limiting portion 34. The expansion limiting portion 34b is configured so that flexibility thereof increases toward a distal end thereof stepwisely. In accordance with an exemplary embodiment, even in this case, the delivery device 10 can have the advantageous effect of the expansion limiting portion 34a according to the first modification example.

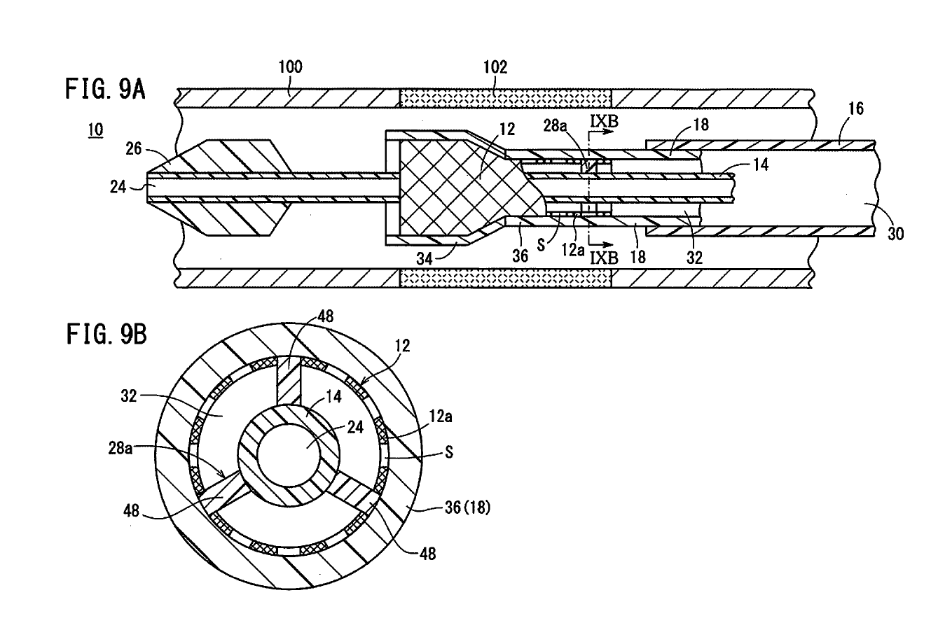

Furthermore, as illustrated in FIGS. 9A and 9B, the delivery device 10 according to the present embodiment may have a stopper portion 28a according to a modification example instead of the stopper portion 28. The stopper portion 28a can have a plurality or multiple (for example, three) locking portions 48 which extend along the radial direction of the shaft 14 and which are arranged so as to be inserted into a gap S (mesh) between struts 12a of the stent 12. The respective locking portions 48 are configured to have a rod shape or a plate shape in a state of being fixedly attached to the outer surface of the shaft 14.

According to the delivery device 10 including the stopper portion 28a according to this modification example, the respective locking portions 48 of the stopper portion 28a are inserted into the gap S between the struts 12a of the stent 12. Therefore, when the inner sheath 18 is moved rearward to the proximal side relative to the shaft 14, the stent 12 can be reliably prevented from being misaligned with the indwelling-targeted portion 102 in the axial direction.

In addition, the stent 12 can expand so that the respective struts 12a slip out from the locking portions 48. Therefore, the possibility that the stopper portion 28a may interfere with the expansion of the stent 12 can be reduced. The stopper portion 28a according to the present modification example, for example, may have one locking portion 48, or may have four or more locking portions 48.

In the present embodiment, the delivery device 10 may be configured so that the stopper portion 28a according to the modification example is combined with the expansion limiting portion 34a according to the first modification example, or is combined with the expansion limiting portion 34b according to the second modification example.

Hitherto, the present invention has been described with reference to the preferred embodiments. However, without being limited to the above-described embodiments, the present invention can be modified in various ways within the scope not departing from the gist of the present invention, as a matter of course.

The detailed description above describes a stent delivery device. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A stent delivery device comprising:
a shaft;
a tubular inner sheath that is arranged on an outer surface side of the shaft so as to be movable relative to the shaft along an axial direction;
a tubular outer sheath that is arranged on an outer surface side of the inner sheath so as to be slidable relative to the inner sheath along the axial direction; and
a stent that is received in a self-expandable state between the shaft and the inner sheath, wherein
a distal portion of the inner sheath has an expansion limiting portion which is configured to be more flexible than the outer sheath and by which at least a distal end of the stent is received,
in a state where the outer sheath is moved rearward and the expansion limiting portion is exposed outward from the outer sheath, the expansion limiting portion limits expansion of the stent so that an outer diameter of the distal end of the stent becomes a first diameter (D1) which is larger than an inner diameter (Da) of the outer sheath, and
in a state where the inner sheath is moved rearward and the stent is exposed outward from the inner sheath, the stent expands so that the outer diameter of the distal end of the stent becomes a second diameter (D2) which is larger than the first diameter (D1).

2. The stent delivery device according to claim 1, wherein
the inner sheath has an inner sheath main body which is disposed on a proximal side relative to the expansion limiting portion and which has greater rigidity than that of the expansion limiting portion, and
when a total length of the expansion limiting portion is set to L, a total length of the stent in a received state is set to L1, and an outer diameter of the stent in a natural state is set to D, an equation of L≦L1−D/2 is satisfied.

3. The stent delivery device according to claim 1, wherein
flexibility of the expansion limiting portion increases continuously or stepwisely toward a distal end.

4. The stent delivery device according to claim 2, further comprising:
a stopper portion which is disposed on the shaft so as to limit displacement along the axial direction of the stent.

5. The stent delivery device according to claim 4, wherein
the stopper portion is located inside the inner sheath main body and engages with the stent in a state where the distal end of the stent is exposed from the expansion limiting portion.

6. The stent delivery device according to claim 5, wherein
the stopper portion is in contact with a proximal end of the stent in a state of being fixed to the shaft.

7. The stent delivery device according to claim 5, wherein
the stopper portion has a locking portion which extends in a radial direction of the shaft and which is arranged to be inserted into a gap (S) between struts of the stent.

8. The stent delivery device according to claim 7, wherein
the stopper portion has a plurality of locking portions, which extend along the radial direction of the shaft and which are arranged so as to be inserted into the gap S between the struts of the stent.

9. The stent delivery device according to claim 7, wherein
the locking portions are configured to have a rod shape or a plate shape in a state of being fixedly attached to the outer surface side of the shaft.
